(19) United States
(12) Patent Application Publication
(10) Pub. No.: US 2008/0221381 A1
(43) Pub. Date: Sep. 11, 2008

(54) Recharge Device for Brachytherapy of Tumors

(76) Inventor: Anwer Puthawala, Buckenhof (DE)

Correspondence Address: JORDAN AND HAMBURG LLP, 122 EAST 42ND STREET, SUITE 4000, NEW YORK, NY, 10168, US

(21) Appl. No.: 11/662,213

(22) PCT Filed: Sep. 9, 2005
(86) PCT No.: PCT/EP05/09691

§ 371 Date: May 1, 2007

(30) Foreign Application Priority Data
Sep. 10, 2004 (DE) 10 2004 043 880.3

(51) Int. Cl. A61N 5/00

(52) U.S. Cl. 600/1

(57) ABSTRACT

A reloading device for brachytherapy of tumors is to be simplified in terms of the replacement of the radiation source at the irradiation site. At least two tubes are provided for receiving a longitudinally displaceable cable at the end of which a radiation source can be applied. The tubes can be connected, via a source replacement device and a shield, to a removable and adjustable distributor, from which lines lead to the tumor. By adjusting the distributor, the respectively desired line can be oriented to the respectively desired tube and in this way the desired radiation source can be introduced preferably step by step into the tumor.

DE 34 42 767 C2 discloses a reloading device for brachytherapy of tumors, with several tubes in which radioactive radiation sources can be moved lengthwise on cables or rods by means of magnets. These radiation sources can be introduced via a connection slide into lines which lead to the tumor.

For each radiation treatment, very specific radiation sources have to be used. A large number of radiation sources are provided in the device, each on a cable, and the radiation sources needed in each case are introduced through the lines into the patient. If, for example, radiation treatment is to be performed with a high or low dose, this is difficult to do with the abovementioned device.

The object of the invention is to take a device of the type mentioned at the outset and simplify it compared to the prior art so that replacement and also a step by step displacement of the radiation source can be done easily and quickly at the treatment site.

According to the invention, this object is achieved by the features of patent claim 1.

In the invention, different radiation sources with different activities can be accommodated in several tubes. By adjusting the distributor, the respectively released radiation source can be introduced into the desired line leading to the patient, without the need for elaborate replacement and exchange procedures.

Advantageous developments of the invention are set out in the dependent claims.

A particularly expedient embodiment is set out in patent claim 7. The curvature of the tubes gives a particularly compact and space-saving structure compared to the prior art in which the tubes are of elongate configuration.

The invention is explained in more detail below with reference to the drawing, in which;

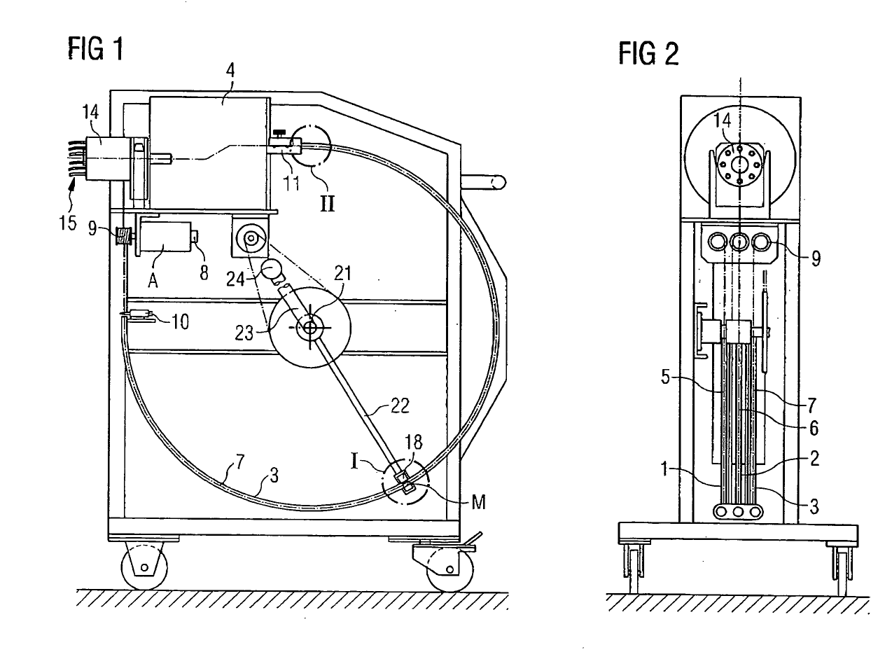

FIGS. 1 and 2 show a side view and a front view of a device according to the invention,

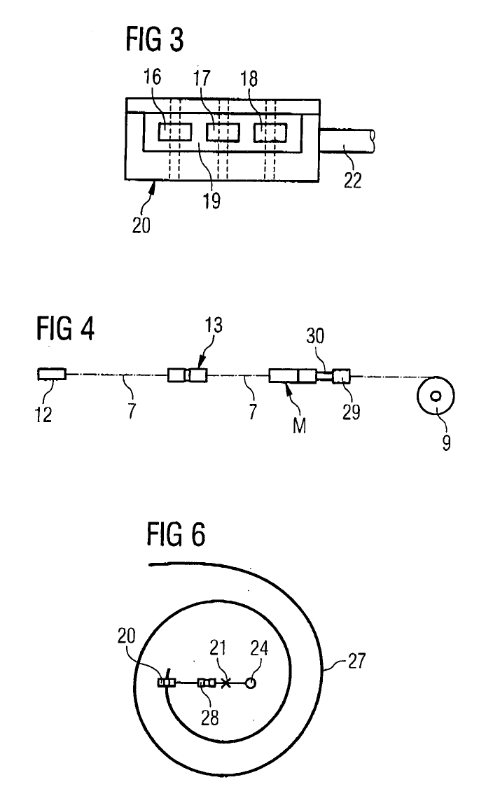

FIGS. 3 and 4 show details from FIG. 1 at positions I and II,

FIG. 5 shows a side view of a variant of the device according to FIGS. 1 to 3, and

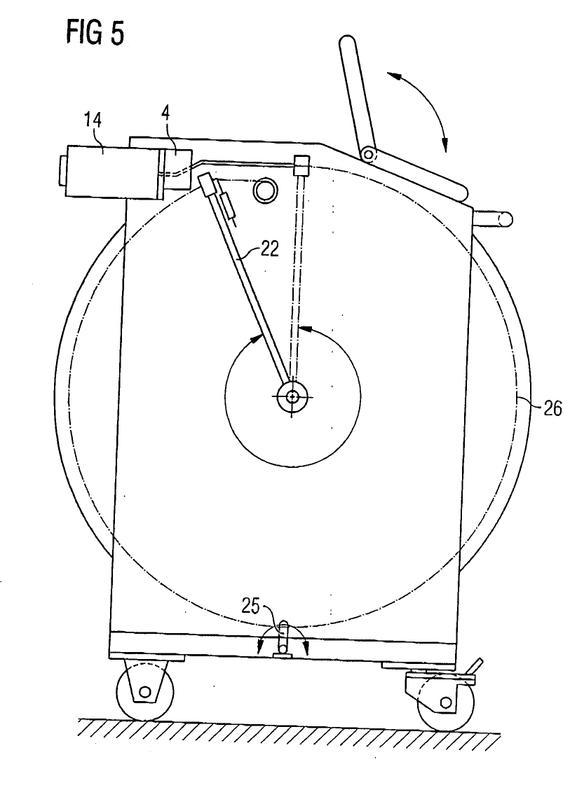

FIG. 6 shows a particularly space-saving design of a device according to the invention.

In FIGS. 1 and 2, reference numbers 1, 2, 3 designate three tubes made of non-magnetizable material, e.g. stainless steel, which are curved in a partial circle, with approximately a quarter circle lying between the two ends of each tube. At the upper end of the reloading device there is a shield 4 to which the upper ends of the tubs 1, 2, 3 are guided. In the inside of the tubes 1, 2, 3, a flexible cable 5, 6, 7 is in each case guided longitudinally displaceably.

The cables 5, 6, 7 are represented by dot-and-dash lines. To measure the distance traveled in each case by the cables 5, 6, 7 and thus by the radiation sources, pulse generators are used, of which one is illustrated in FIG. 1 and designated by reference number 8. The pulse generators are connected to drive motors, of which the drive motor A can be seen in FIG. 1. The cables 5, 6, 7 are wound helically onto reel elements. The reel element of the pulse generator 8 is designated in FIG. 1 by reference number 9. For each cable 5, 6, 7, there is an electrical clamp device which securely holds the cable that is not, to be adjusted, and of which the clamp device 10 for the cable 7 is shown in FIG. 1. Between the shield device 4 and the upper ends of the tubes 1, 2, 3, there is a replacement device 11 for the radiation sources. The radiation sources, of which one is indicated and designated by reference number 12 in FIG. 4, are applied at the upper ends of the cables 5, 6, 7, e.g. two radiation sources 12 of the cables 5 and 7. FIG. 4 shows that the respective radiation source, e.g. 12, is connected to the respective cable, e.g. 7, via a screw connection 13, of which one part is mounted rotatably on the end of the cable 7 and of which the other part is mounted on a cable section that supports the radiation source 12. Instead of the screw connection, a plug connection can also be provided.

A distributor 14 has lines 15 which can be introduced via coupling pieces into the tumor to be treated. It is mounted detachably on the shield 4 so that it can be carried by the patient in radiation treatment intervals. The distributor 14 is adjustable (rotatable) and the inlet of the desired line 15 can be oriented to the outlet of one of the tubes 1, 2, 3. The desired radiation source 12 or a dummy can thus be introduced into the distributor 14. The shield 4 and the tubes 1, 2, 3 are oriented such that the cables 5, 6, 7 are guided approximately horizontally to the distributor 14.

For longitudinal displacement of the cables 5, 6, 7, the tubes 1, 2, 3 are surrounded by permanent magnets 16, 17, 18 (FIG. 3) with central apertures, which are coupled magnetically to magnet elements made of magnetizable material on the cables 5, 6, 7 and can be moved on the tubes 1, 2, 3. In FIG. 1, the magnet element of the cable 7 is designated by M. Another magnet configuration, e.g. two flat magnets, can also satisfy the desired function.

The permanent magnets 16, 17, 18 (FIG. 3) are mounted in the hollow space 19 of a holder 20 secured at the end of an arm 22 that can rotate about the center 21 of the circle of the tubes 1, 2, 3. In order to compensate for curvature tolerances of the tubes 1, 2, 3, the permanent magnets 16, 17, 18 are mounted in the holder 20 so as to be able to move in two dimensions transversely with respect to the longitudinal direction of the tube. A compensating weight 24 is fitted on an extension 23 of the arm 22 protruding beyond the pivot point of the arm 22, so that an exact step by step movement of the respective radiation source is possible.

Different radiation sources are mounted in the tubes 1, 2, 3. The tumor treatment involves introducing one of the lines 15 via a coupling piece into the tumor and setting the distributor 14 so that the desired radiation source can be introduced into the inlet selected by setting the distributor 14 for this line. The arm 22 is then turned counterclockwise by motor, only the cable with the correct radiation source being carried along with it, because the other cables are arrested by the respective clamp devices 10 (not shown in FIG. 2). For example, a radiation source for a high dose, one for a low dose, and a dummy (non-irradiating pin) for test purposes c n be accommodated in the tubes 1, 2, 3. In the example, the dummy can be connected to the end of the cable 6. For the dummy, a pulse generator 8 and a drive motor are not absolutely essential. A radiation source for pulse dose can also alternately be provided, permitting repeat irradiation with radiation-free intervals. The particular travel of the respectively required radiation source is detected by the respective pulse generator, so that the radiation source can be brought to the correct position in the tumor and can also be moved exactly the desired number of steps therein.

The drive motors A serve to withdraw the respective radiation source from the patient in the event of current failure, since they are battery-powered. They also generate a pretensioning of the cables 5, 6, 7 and keep them taut.

The structure according to FIGS. 1 and 2 shows that the tubes 1, 2, 3 protrude laterally from the device on the right.

FIG. 5 shows a symmetrical structure in relation to the carriage arrangement in which there is approximately an eighth part of a circle lying between the ends of each tube. Not all parts of the device are shown and labeled in FIG. 5. A finger-shaped tube holder 25 is here arranged on the underside of the tubes 1, 2, 3 (diagrammatically embodied by the broken line 26) and this tube holder 25 engages round the tubes 1, 2, 3. It can be folded aside so that the holder 20 can be moved past.

A particularly compact structure is shown in FIG. 6. Here, a spiral tube 27 is shown diagrammatically. Instead of the rigid arm 22, an arm with a slide element 28 is provided which in terms of its length can be adapted to the respective radius of the spiral tube 27. Instead of a spiral tube, a helical tube can also be provided.

FIG. 4 shows that a pin 29 made of non-magnetizable material is connected to the magnet element M on the cable 7 and has a notch 30 into which the clamp device 10 engages.

1. A reloading device for brachytherapy of tumors, comprising at least two tubes for receiving in each tube a longitudinally displaceable cable, each cable being adopted to have a radiation source attached to one end portion thereof;
a removable distributor comprising a plurality of lines extending through the distributor and adapted to be guided into a tumor, each line having an inlet opening; and
a shield;
wherein the distributor is detachably arranged on one side of the shield with the tubes being arranged on the opposite side of the shield, and wherein the distributor is adjustable to permit the respectively desired radiation source to be introduced into the inlet opening of the respectively desired line.

2. The reloading device as claimed in claim 1, further comprising a source replacement device arranged about the end of the tubes directed toward the shield.

3. The reloading device as claimed in claim 2, wherein the radiation source has a cable section extending from one end thereof and is connected to the respective cable via a screw connection, and wherein one part of the screw connection is mounted rotatably on one of the cable and the cable section.

4. The reloading device as claimed in claim 1, wherein each cable is assigned a pulse generator at the end remote from the shield, to which a reel element supporting the cable is connected and which delivers a predetermined number of pulses per revolution.

5. The reloading device as claimed in claim 4, wherein the pulse generator is connected to a drive motor.

6. The reloading device as claimed in claim 4, wherein a clamp device is disposed at a distance from an inlet opening of each tube, and wherein the clamp device is adapted to securely hold the cable that is not to be adjusted.

7. The reloading device as claimed in claim 1, wherein the tubes are curved in a partial circle.

8. The device as claimed in claim 7, wherein the shield is arranged at an upper end of the reloading device and guides the cables approximately horizontally to the distributor.

9. The reloading device as claimed in claim 7, wherein each tube is surrounded by a magnet which can be moved along the tube and which acts on a magnetically coupable magnetic element on the associated cable to entrain the cable as the cable moves within the tube.

10. The reloading device as claimed in claim 9, wherein the magnets are mounted in a common holder secured at the end of an arm that can rotate about a center of curvature of the tubes.

11. The reloading device as claimed in claim 10, wherein in order to compensate for curvature tolerances of the tubes, the magnets are mounted in the holder so as to be able to move transversely with respect to a longitudinal direction of the tubes.

12. The reloading device as claimed in claim 10, wherein a compensating weight is fitted on an extension of the arm protruding beyond a pivot point of the arm.

13. The reloading device as claimed in claim 7, wherein a tube holder is provided about a lower portion of the tubes to secure the tubes to a housing of the device.

14. The reloading device as claimed in claim 7, wherein the distance between the two ends of each tube is approximately a quarter circle.

15. The reloading device as claimed in claim 7, wherein the distance between the two ends of each tube is approximately an eighth part of a circle.

16. The reloading device as claimed in claim 1, wherein the tubes have a spiral configuration.

17. The reloading device as claimed in claim 7, wherein a dummy is accommodated in one of the tubes.
